(19) United States
(12) Patent Application Publication
(10) Pub. No.: US 2024/0407744 A1
(43) Pub. Date: Dec. 12, 2024

(54) X-RAY DIAGNOSIS APPARATUS AND CONSOLE APPARATUS

(71) Applicant: Canon Medical Systems Corporation, Otawara-shi (JP)

(72) Inventors: Haruki IWAI, Otawara (JP); Norimitsu KOSUGI, Nasushiobara (JP); Yoshinori SHIMIZU, Nasushiobara (JP); Hiroki TONOTSUKA, Otawara (JP); Makoto ISHII, Nasushiobara (JP); Seiichirou NAGAI, Otawara (JP); Hiroyoshi KOBAYASHI, Nasushiobara (JP); Toshikatsu OOHASHI, Otawara (JP)

(73) Assignee: Canon Medical Systems Corporation, Otawara-shi (JP)

(21) Appl. No.: 18/731,668

(22) Filed: Jun. 3, 2024

(30) Foreign Application Priority Data
Jun. 8, 2023 (JP) 2023-094854

(51) Int. Cl. A61B 6/46; A61B 6/00; A61B 6/06; G06F 3/04845; G06F 3/04847

(52) U.S. Cl. CPC A61B 6/469; A61B 6/06; A61B 6/465; A61B 6/54; G06F 3/04845; G06F 3/04847

(57) ABSTRACT

An X-ray diagnosis apparatus includes: an X-ray; an X-ray detector; processing circuitry configured to generate an X-ray image based on the detected X-rays, and causes a display to display the X-ray image; a first operator that receives an operation of inputting a central position for a displayed image to be displayed on the display; and a second operator that receives an operation of inputting a field-of-view size of the displayed image in a stepless manner. The processing circuitry is configured to change the field-of-view size of the displayed image in response to an operation at the second operator, while the central position of a current displayed image is being maintained.

### CROSS-REFERENCE TO RELATED APPLICATION(S)

This application is based upon and claims the benefit of priority from Japanese Patent Application No. 2023-094854, filed Jun. 8, 2023, the entire contents of which are incorporated herein by reference.

### FIELD

Embodiments described herein relate generally to an X-ray diagnosis apparatus and a console apparatus.

### BACKGROUND

With a conventional technique using an X-ray diagnosis apparatus, a user performs the technique while looking at an image of a desired target position (area of interest) within the X-ray detector according to a clinical purpose, not an X-ray image acquired from the entire surface of the X-ray detector. In this case, the user moves the X-ray irradiation range (irradiation field) to a desired target position within the X-ray detector through a stick control. At this time, the X-ray irradiation range is control by an asymmetrical control of four (top, bottom, left, and right) diaphragm blades so that only the target position is irradiated with X-rays.

In such an X-ray diagnosis apparatus, only a fixed stepwise discrete size can be selected as a field-of-view size for displaying the target position. For this reason, in order to display the target position in a clinically desirable field-of-view size, a fixed size that is close to and larger than a desired field-of-view size is selected, and the X-ray field-of-view size and the X-ray irradiation range are adjusted by narrowing the irradiation range to the selected fixed size using the X-ray diaphragm. As only a predefined fixed size is selectable and the selected fixed size is displayed in full screen, the field-of-view size cannot be easily and freely set to a preferable size, and it is therefore difficult to display a target position in a desired size. For this reason, a user may not be able to control the field of view simply and easily when controlling the X-ray irradiation range in the X-ray detector, and this may affect efficiency in examination.

### BRIEF DESCRIPTION OF THE DRAWINGS

FIG. 1 is a drawing showing an example of a configuration of an X-ray diagnosis apparatus according to a first embodiment.

FIG. 2 is a diagram illustrating the configuration of an input interface according to the first embodiment.

FIG. 3 is a flowchart illustrating a processing procedure of the X-ray diagnosis apparatus according to the first embodiment.

FIG. 4 is a diagram explaining a process of changing a field-of-view range of a displayed image in the first embodiment.

FIG. 5 is a diagram showing a central position changed from that in FIG. 4.

FIG. 6 is a diagram showing an irradiation range changed from that in FIG. 5.

FIG. 7 is a diagram showing a field-of-view size changed from that in FIG. 6.

FIG. 8 is a diagram showing the field-of-view range further changed from that in FIG. 7.

FIG. 9 is a flowchart illustrating a processing procedure of the X-ray diagnosis apparatus according to a second embodiment.

FIG. 10 is an example of a display screen displayed by the X-ray image diagnosis apparatus according to the second embodiment.

FIG. 11 is a diagram showing that the field-of-view range of an enlarged image exceeds the irradiation range in the second embodiment.

FIG. 12 is a diagram showing an irradiation range changed from that in FIG. 11.

FIG. 13 is a diagram showing an irradiation range enlarged from that in FIG. 11.

FIG. 14 is a diagram showing that the field-of-view range of an enlarged image exceeds the range in which an X-ray detector is set in the second embodiment.

FIG. 15 is a diagram showing a reduced field-of-view range of FIG. 14.

FIG. 16 is a diagram showing an example of a display screen displayed under the state shown in FIG. 14.

FIG. 17 is a diagram showing a display of an X-ray image including the range in which an X-ray detector is set, as a whole image in a second embodiment.

### DETAILED DESCRIPTION

In general, according to one embodiment, an X-ray diagnosis apparatus includes: an X-ray tube that generates X-rays; an X-ray detector that detects the X-rays; processing circuitry configured to generate an X-ray image based on the detected X-rays, and causes a display to display the X-ray image; a first operator that receives an operation of inputting a central position for a displayed image to be displayed on the display; and a second operator that receives an operation of inputting a field-of-view size of the displayed image in a stepless manner. The processing circuitry is configured to change the field-of-view size of the displayed image in response to an operation at the second operator, while the central position of a current displayed image is being maintained.

Hereinafter, the embodiments of the X-ray diagnosis apparatus will be explained in detail with reference to the accompanying drawings. In the description hereinafter, structural elements having substantially the same functions and configurations will be denoted by the same reference symbols, and a duplicate description of such elements will be given only where necessary.

### First Embodiment

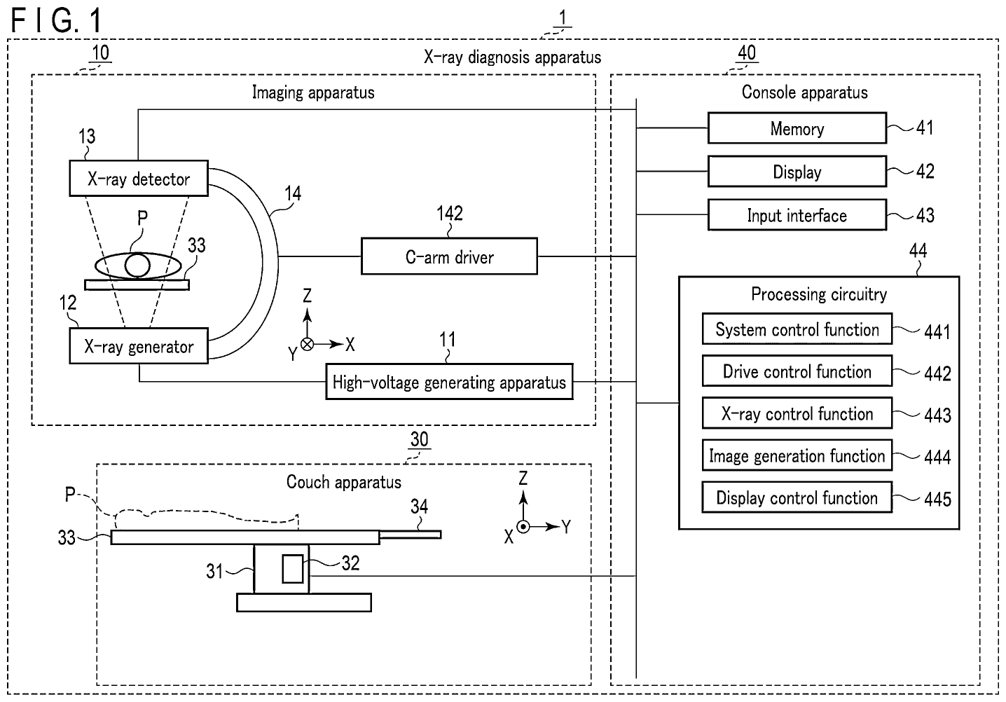

FIG. 1 is a drawing showing a configuration example of an X-ray diagnosis apparatus 1 according to the first embodiment. The X-ray diagnosis apparatus 1 corresponds to, for example, an X-ray fluoroscopy imaging diagnosis apparatus used in a contrast examination of a digestive tract. The X-ray diagnosis apparatus 1 may be, for example, an X-ray fluoroscopy imaging diagnosis apparatus for a circulatory system used in a contrast examination of blood vessels.

As shown in FIG. 1, the X-ray diagnosis apparatus 1 has an imaging apparatus 10, a couch apparatus 30, and a console apparatus 40. The imaging apparatus 10 has a high voltage generating apparatus 11, an X-ray generator 12, an X-ray detector 13, a C-arm 14, and a C-arm driving apparatus 142.

The high voltage generating apparatus 11 generates a high voltage to be applied between an anode and a cathode, and outputs the high voltage to an X-ray tube, so that thermoelectrons generated from the cathode of the X-ray tube are accelerated.

The X-ray generator 12 has an X-ray tube that irradiates a subject P with X-rays, a plurality of filters having a function of attenuating or reducing an amount of irradiation X-rays (hereinafter “added filters”), and an X-ray diaphragm.

The X-ray tube is a vacuum tube that generates X-rays. The X-ray tube has a tube bulb, a filament (cathode) provided on the tube bulb, and a tungsten anode. The X-ray tube accelerates the thermoelectrons released from the filament by the high voltage. The X-ray tube generates X-rays by making the accelerated electrons collide with the tungsten anode.

The X-ray diaphragm is located between the X-ray tube and the X-ray detector 13, and is made of a lead plate, which serves as a metal plate. The X-ray diaphragm shields against X-rays outside an opening area, thereby limiting the X-rays generated by the X-ray tube and irradiating only a region of interest of the subject P with the limited X-rays, so that a size of an X-ray irradiation range (hereinafter, a “field-of-view size”) is adjusted. For example, the X-ray diaphragm has four diaphragm wings that are separately movable, and adjusts an area shielded from X-rays and, in turn, a field-of-view size, to a desired size by sliding these diaphragm wings. The diaphragm wings of the X-ray diaphragm are driven by a driving apparatus (diaphragm driver) (not shown) in accordance with the region of interest that has been input by the operator through the input interface 43.

As described above, the X-ray diaphragm limits the irradiation range of X-rays generated by the X-ray tube. By limiting the X-ray irradiation range by the X-ray diaphragm, it is possible to irradiate, with X-rays, only an imaging region (or imaging range) of a subject P as desired by an operator. In other words, the X-ray diaphragm can inhibit a part (or range) differing from the photographed part (or photographed range) from being unnecessarily exposed to X-rays. Furthermore, the irradiation range to be irradiated with X-rays can be freely set by controlling the driving of four diaphragm wings. The X-ray diaphragm can reduce scattered X-rays and remove X-rays that are out of the focal point. Hereinafter, the phrase “limiting the X-ray irradiation range” may be read as “stopping X-rays”, “focusing X-rays”, or “limiting the X-ray irradiation range”.

The X-ray detector 13 detects X-rays that have been emitted from the X-ray tube and have passed through the subject P. As the X-ray detector 13, both an X-ray detector capable of directly converting X-rays into electric charge and an X-ray detector capable of converting X-rays into light then into electric charge can be adopted, and the former detector will be described hereinafter as an example; however, the latter detector can also be adopted. In other words, the X-ray detector 13 has a flat panel detector (FPD) that converts X-rays that have passed through the subject P into electric charge and accumulates the electric charge, and a gate driver that generates a drive pulse for reading the electric charge accumulated in this FPD. The FPD is comprised of micro detection elements, which are two-dimensionally arranged in a row direction and a line direction. Each of the detection elements has a photoelectric film that senses X-rays and generates electric charge in accordance with an amount of incident X-rays, an electric charge accumulating capacitor that accumulates electric charge generated on the photoelectric film, and a TFT (thin-film transistor) that outputs the electric charge accumulated on the electric charge accumulating capacitance at a predetermined timing. The accumulated electric charge is sequentially read by a drive pulse supplied by the gate driver. The X-ray detector 13 is an example of an X-ray detector. In the stage after the X-ray detector 13, projection data generating circuitry (not shown) is provided. The projection data generating circuitry includes a parallel-serial converter for converting a digital signal read in parallel from the FPD of the X-ray detector 13 in units of rows or units of columns into a chronological serial signal (chronological projection data). The chronological projection data is output from the projection data generating circuitry and supplied to the console apparatus 40.

The C-arm 14 may be configured to retain the X-ray generator 12 and the X-ray detector 13 and perform X-ray imaging while rotating. The C-arm 14 has a structure that allows the C-arm 14 to hold the X-ray generator 12 and the X-ray detector 13 in such a manner that they are opposed to each other, with the subject P and the top plate 33 being interposed therebetween, so that X-ray imaging of the subject P who lays on the top plate 33 can be performed. The C-arm 14 is slidably and rotatably supported with respect to each of the plurality of rotation axes. The C-arm 14 is provided with a plurality of power sources at locations suitable for realizing operations of sliding or rotation. These power sources constitute a C-arm driving apparatus 142. The C-arm driving apparatus 142 reads a drive signal from a drive control function 442 to cause the C-arm 14 to slide, rotate, or move linearly. The C-arm 14 is an example of a support arm.

The couch apparatus 30 is a device to place the subject P and to move the subject P thereon, and includes a base 31, a couch driving apparatus 32, a top plate 33, and a support frame 34.

The base 31 is a housing placed on the floor and supporting the support frame 34 movably in the vertical direction (a Z-axis direction).

The couch driving apparatus 32 is a motor or actuator that is stored in the housing of the couch apparatus 30 and moves the top plate 33 on which the subject P is placed in the longitudinal direction of the top plate 33 (a Y-axis direction). The couch driving apparatus 32 reads a drive signal from the drive control function 442, and moves the top plate 33 in a horizontal direction or a vertical direction with respect to the floor. The position relationship between the subject P and the imaging axis changes when the C-arm 14 or the top plate 33 moves. The couch driving apparatus 32 may move not only the top plate 33 but also the support frame 34 in the longitudinal direction of the top plate 33.

The top plate 33 is a plate provided on the top surface of the support frame 34 and on which the subject P is placed.

The support frame 34 is provided above the base 31, and slidably supports the top plate 33 in its longitudinal direction.

In the couch apparatus 30, the top plate 33 may be movable with respect to the support frame 34, or the top plate 33 and the support frame 34 may be together movable with respect to the base 31.

The console apparatus 40 includes a memory 41, a display 42, an input interface 43, and processing circuitry 44. Hereinafter, the console apparatus 40 will be described as a device separate from the imaging apparatus 10; however, the console apparatus 40 or some of the structural elements thereof may be incorporated in the imaging apparatus 10. The console apparatus 40 is, for example, a medical image processing apparatus.

Hereinafter, the console apparatus 40 will be described as an apparatus performing a plurality of functions with a single console; however, it is possible to perform a plurality of functions with separate consoles. For example, the functions of the processing circuitry 44, such as an image generation function 444 (described later) may be implemented on different console devices in a distributed manner.

The memory 41 is a storage device such as a hard disk drive (HDD), a solid state drive (SSD), or an integrated circuit storage unit, etc., configured to store various kinds of information. The memory 41 may be, aside from the HDD, SSD or the like, a portable storage medium such as a CD (compact disc), a DVD (digital versatile disc) or a flash memory. Alternatively, the memory 41 may be a drive apparatus that writes and reads various types of information to and from a semiconductor memory, such as a flash memory or a random access memory (RAM), etc. The storage area of the memory 41 may be in the console apparatus 40, or in an external storage device connected via a network.

The memory 41 stores programs executed by the processing circuitry 44 and various types of data used for the processing in the processing circuitry 44. As the programs, programs that can be installed onto a computer from a network or a non-transitory computer readable storage medium and that cause the computer to realize the functions of processing circuitry 44 are used. Various types of data used in the present description are typically digital data. The memory 41 is an example of a storage unit.

The display 42 displays various kinds of information. For example, the display 42 outputs medical images (X-ray images) generated by the processing circuitry 44, and a graphical user interface (GUI) or the like for receiving various types of operations from the operator. For example, the display 42 is a liquid crystal display or a CRT (cathode ray tube) display. The display 42 may be provided on the imaging apparatus 10. The display 42 may be a desktop type, or comprised of a tablet device, etc. capable of wireless communication with the main body of the console apparatus 40. The display 42 is an example of a display.

The input interface 43 accepts various kinds of input operations from the operator, converts the accepted input operations to electric signals, and outputs the electric signals to the processing circuitry 44. For example, the input interface 43 receives scan conditions for collecting projection data, instructions for moving the C-arm 14, setting a region of interest (ROI), operations for performing fluoroscopy imaging, and so on from an operator. For example, the input interface 43 is realized by a mouse or a keyboard for performing various processing etc. of the processing circuitry 44, a track ball, a switch button, a joystick, a touch screen in which a display screen and a touch pad are integrated, or a non-contact input circuit using an optical sensor, or an audio input circuit, etc. The input interface 43, which is connected to the processing circuitry 44, converts an input operation received from an operator into an electric signal, and outputs the electric signal to the control circuit. In the present specification, the input interface is not limited to physical operating components such as a mouse and a keyboard. Examples of the input interface include processing circuitry of an electric signal, which receives an electric signal corresponding to an input operation from an external input device, which is provided separately from the apparatus, and outputs the received electric signal to the processing circuitry 44. The input interface 43 may be provided in the imaging apparatus 10 and configured as a tablet device capable of communicating wirelessly with the console apparatus 40. The input interface 43 is an example of an input unit.

The input interface 43 includes a first operator (a first operation unit) for receiving an operation to input a central position of a displayed image to be displayed on the display 42 and a second operator (a second operation unit) for receiving an operation of steplessly inputting a field-of-view size of the displayed image. The second operator is, for example, an operation unit that allows a reception of a move operation that is input by a user. In this case, the second operator receives an input of the field-of-view size based on the move operation that is input by a user. The second operator may receive an input regarding a reduction of the field-of-view size in accordance with a move operation that is input by a user for a movement in a first direction, and may receive an input regarding an enlargement of the field-of-view size in accordance with a move operation that is input by a user for a movement in first direction. The second operator may be a slide bar configured to receive an input of a field-of-view size in accordance with an amount of movement of the slide bar by the user. The second operator may further receive an operation of canceling an input of a field-of-view size in addition to an operation of inputting a field-of-view size.

The processing circuitry 44 controls the entire operation of the X-ray diagnosis apparatus 1. The processing circuitry 44 is a processor that invokes a program in the memory 41 and performs a system control function 441, a drive control function 442, an X-ray control function 443, an image generation function 444, and a display control function 445.

FIG. 1 illustrates the case where the system control function 441, the drive control function 442, the X-ray control function 443, the image generation function 444, and the display control function 445 are realized in single processing circuitry 44; however, the embodiment is not limited to this example. For example, processing circuitry may be configured by combining a plurality of independent processors, and the processors may execute respective programs to implement the functions. The system control function 441, the drive control function 442, the X-ray control function 443, the image generation function 444, and the display control function 445 may be respectively referred to as system control circuitry, drive control circuitry, X-ray control circuitry, image generation circuitry, and display control circuitry; furthermore, each of those functions may be implemented as individual hardware circuitry. The above description of each of the functions implemented by the processing circuitry 44 is applicable to the subsequent embodiments and modifications.

Hereinafter, the console apparatus 40 will be described as an apparatus performing a plurality of functions with a single console; however, it is possible to perform a plurality of functions with separate apparatuses. For example, the functions of the processing circuitry 44 may be implemented on different apparatuses in a distributed manner.

The term “processor” used in the above explanation means, for example, circuitry such as a CPU (central processing unit), a GPU (graphics processing unit), an ASIC (application specific integrated circuit), or a programmable logic device (for example, an SPLD (simple programmable logic device), a CPLD (complex programmable logic device), or an FPGA (field programmable gate array)). If the processor is a CPU, for example, the processor realizes its function by reading and executing the program stored in the storage circuitry. If the processor is for example an ASIC, on the other hand, the function is directly implemented in a circuit of the processor as a logic circuit, instead of storing a program in a storage circuit. Each processor of the present embodiment is not limited to a case where each processor is configured as a single circuit; a plurality of independent circuits may be combined into one processor to realize the function of the processor. In addition, a plurality of structural elements in FIG. 1 may be integrated into one processor to realize the function. The above description of the “processor” is applicable to the subsequent embodiments and modifications.

The apparatus constituted by the memory 41, the display 42, the input interface 43, and the image generation function 444 and the display control function 445 of the processing circuitry 44 may be called a “medical image processing apparatus”. For this reason, the descriptions of the memory 41, the display 42, the input interface 43, and the image generation function 444 and the display control function 445 of the processing circuitry 44 serve as the description of the medical image processing apparatus. The medical image processing apparatus constituted by the memory 41, the display 42, the input interface 43, and the image generation function 444 and the display control function 445 of the processing circuitry 44 may be provided as a separate apparatus communicable with the X-ray diagnosis apparatus 1.

The processing circuitry 44 controls, through the system control function 441, each of the plurality of structural elements of the X-ray diagnosis apparatus 1 based on an input operation received from the operator via the input interface 43. For example, the processing circuitry 44 controls the structural elements of the imaging apparatus 10 in accordance with imaging conditions.

The processing circuitry 44 controls, through realization of the drive control function 442, the C-arm driving apparatus 142 and the couch driving apparatus 32 based on, for example, information regarding the driving of the C-arm 14 and the top plate 33, which is input from the input interface 43. The processing circuitry 44 controls the driving of the X-ray diaphragm by the system control function 442. At this time, the processing circuitry 44 controls the irradiation range in accordance with an operation at the first operator or the second operator. The processing circuitry 44 that enables the drive control function 442 is an example of a drive control unit.

The processing circuitry 44, through the X-ray control function 443 reads, for example, the information from the system control function 441, and controls the X-ray conditions, such as a tube current, a tube voltage, a focal-spot size, an irradiation time, and a pulse width, etc. in the high voltage generating apparatus 11. The X-ray control function 443 reads information from the system control function 441 and controls the driving of the diaphragm wings of the X-ray diaphragm in an asymmetrical manner to control the irradiation range of X-rays. The processing circuitry 44 that enables the X-ray control function 443 is an example of an X-ray control unit.

The processing circuitry 44 acquires, through realization of the image generation function 444, projection data generated based on detection data read from the X-ray detector 13, and generates an X-ray image, such as a fluoroscopy image, based on the projection data. The processing circuitry 44 may perform various types of synthesis processing or subtraction processing on the generated X-ray image. The processing circuitry 44 that enables the image generation function 444 is an example of an image generating unit.

The processing circuitry 44 reads, through realization of the display control function 445, a signal from the system control function 441, and displays a desired X-ray image obtained from the memory 41 on the display 42. The processing circuitry 44 that enables the X-ray control function 445 is an example of a display control unit.

The processing circuitry 44 controls, through realization of the display control function 445, the field-of-view range of a displayed image to be displayed on the display 42 based on an operation at the first operator for controlling the central position of the displayed image and an operation at the second operator for controlling the field-of-view size of the displayed image in a stepless manner. The processing circuitry 44 changes, through realization of the display control function 445, the field-of-view size of the displayed image in accordance with an operation at the second operator, with the central position of the current displayed image maintained. At this time, the processing circuitry 44 drives the X-ray diaphragm in accordance with the change in the field-of-view size to change the X-ray irradiation range in accordance with the change in the field-of-view size.

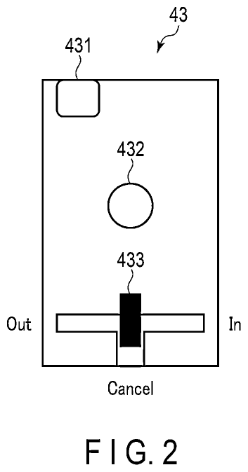

Next, an example of the configuration of the input interface 43 is specifically described. FIG. 2 is a diagram showing the configuration of the input interface 43 in the present embodiment. As shown in FIG. 2, the input interface 43 of the present embodiment includes a start button 431, a central position controller 432, and a field-of-view size controller 433.

The start button 431 is an interface for inputting an operation to start X-ray imaging, such as fluoroscopy. After the start button 431 is operated, an X-ray image generated by X-ray imaging is displayed on the display 42. The start button 431 may be used as an interface for inputting an operation to finish X-ray imaging.

Although not shown, the input interface 43 includes a fixed field-of-view size selecting unit for selecting one from multiple predefined fixed field-of-view sizes (hereinafter, “fixed field-of-view sizes”). The fixed field-of-view sizes are multiple predefined fixed stepless field-of-view sizes. The fixed field-of-view size selecting unit can be realized by, for example, a button.

The central position controller 432 is an interface for inputting the central position of the display screen. The central position controller 432 corresponds to the first operator. The central position controller 432 can be realized by a joystick, a shift lever, a paddle lever, a slide bar, a button, a pedal, a dial, and so on. In the example of FIG. 2, the central position controller 432 is realized by a joystick with which the central position 711 can be controlled in a stepless manner. The central position controller 432 may be an interface to which a coordinate of the central position 711 can be directly input.

The field-of-view size controller 433 is an interface for inputting the field-of-view size of the display screen. The field-of-view size controller 433 corresponds to the second operator. An operation of reducing the field-of-view size corresponds to an operation of zooming in the display screen. An operation of enlarging the field-of-view size corresponds to an operation of zooming out the display screen. The field-of-view size controller 433 is configured in such a manner that the field-of-view size can be freely input in a stepless manner. The field-of-view size to be input may be a relative size, such as an enlargement rate or a reduction rate of the current field-of-view size, or an absolute size, such as the actual field-of-view size. The field-of-view size controller 433 may be an interface to which a field-of-view size can be directly input.

The field-of-view size controller 433 can be realized by a joystick, a shift lever, a paddle lever, a slide bar, a button, a pedal, a dial, a keypad controller, software such as a GUI, and so on. The field-of-view size controller 433 may be realized by a seesaw-type, a twist-type, or ring-type mouse. For example, if a lever is used as the field-of-view size controller 433, the field-of-view size can be input in a stepless manner in accordance with a position or a twist angle of the lever.

In the example of FIG. 2, the field-of-view size controller 433 is realized by a slide bar capable of controlling the field-of-view size in a stepless manner with the position of the bar. For example, if the slide bar is moved to the right side, the field-of-view size is reduced (zoomed in), and if the slide bar is moved to the left side, the field-of-view size is enlarged (zoomed out). If the slide bar is pressed inside the concave part in the center, the enlargement or reduction of the field-of-view size is canceled.

The field-of-view size controller 433 may be integrated with another interface, such as the central position controller 432, etc. For example, a button may be added as the field-of-view size controller 433 to the lever provided as the central position controller 432. A dial combined with other functions may be used as the field-of-view size controller 433. If a keypad-type controller is used as the central position controller 432 and the field-of-view size controller 433, the position of the display area is controlled by a touch position, and the field-of-view size can be enlarged or reduced by pinching. The function of the field-of-view size controller 433 may be integrated into the fluoroscopy pedal. In this case, the field-of-view size can be controlled in a stepless manner in accordance with how much the fluoroscopy pedal is depressed. The field-of-view size may be enlarged or reduced by tilting the joystick while it is being pressed in. In this case, the enlargement or reduction of the field-of-view size can be canceled. The field-of-view size may be enlarged or reduced by placing the joystick into a “hold” state after double clicking.

An already existing structure may be used as the field-of-view size controller 433. For example, an already existing button provided in a lever serving as the central position controller 432 may have a function as the field-of-view size controller 433. In this case, the second operator is a button provided in the first operator, and the processing circuitry 44 determines, through realization of the display control function 445, an amount of change in the field-of-view size of the displayed image in accordance with how the button is pressed by the user and an input operation to the first operator. For example, if the button is pressed once and the pressed state is put on hold for a time longer than a predetermined length of time, the processing circuitry 44 reduces (zooms in) the field-of-view size through the display control function 445, and if the button is pressed twice and the pressed state is put on hold for a time longer than a predetermined length of time after the second press, the processing circuitry 44 enlarges (zooms out) the field-of-view size. The fluoroscopy pedal may be configured to serve the function of the field-of-view size controller 433. The function of the field-of-view size controller 433 may be performed through an audio operation. The field-of-view size may be enlarged or reduced by tilting the lever in the upward or downward direction, while the button is being pressed in. In this case, the field-of-view size is reduced (zoomed in) if the lever is tilted in the upward direction (viewed from the user side), and the field-of-view size is enlarged (zoomed out) if the lever is tilted in the downward direction. The button is preferably pressed down by a user's thumb, and the button is preferably provided in the side part or the upper end of the lever. If the button is provided in the side part of the lever, the button may be provided on the left side if the lever is for a right-handed user, and the button may be provided on the right side if the lever is for a left-handed user.

In addition to the structure, the diaphragm lever or the operation paddle may be provided to control the aspect ratio of the field-of-view size. Multiple functions of the foregoing plurality of operators may be integrated into one operator.

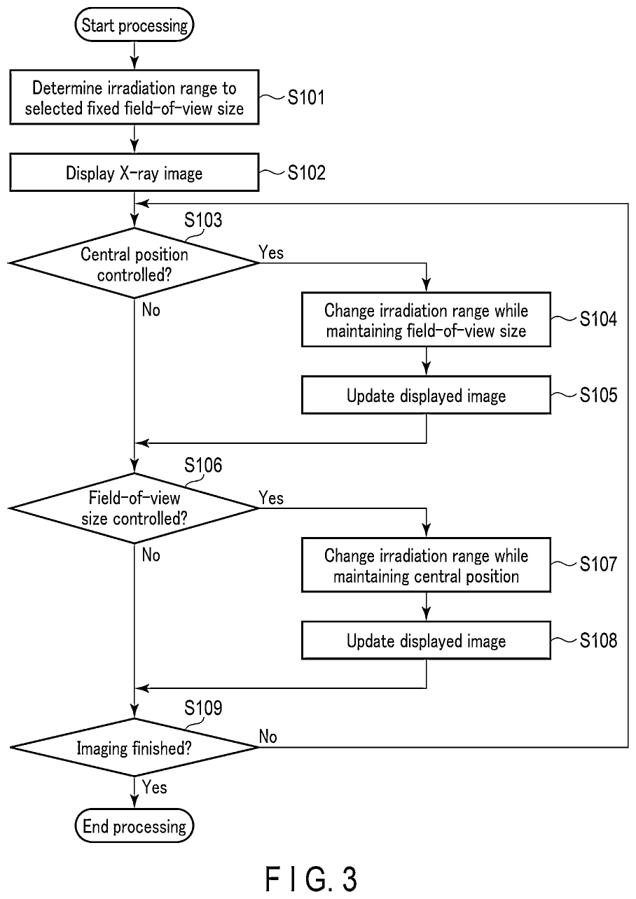

Next, the operation of the X-ray diagnosis apparatus 1 according to the present embodiment is described. FIG. 3 is a flowchart illustrating a processing procedure of the processing performed by the processing circuitry 44 during X-ray imaging. The processing procedure described below is merely an example, and the processing can be changed as far as possible. Omission, replacement, or addition of a step in the process procedure described hereinafter can be made as appropriate, in accordance with an actual situation where the present embodiment is realized.

When X-ray imaging is commenced, the X-ray diagnosis apparatus 1 commences X-ray imaging using the imaging apparatus 10 in response to an operation of the start button 431 shown in FIG. 2 by the user. The X-ray imaging is fluoroscopy, for example.

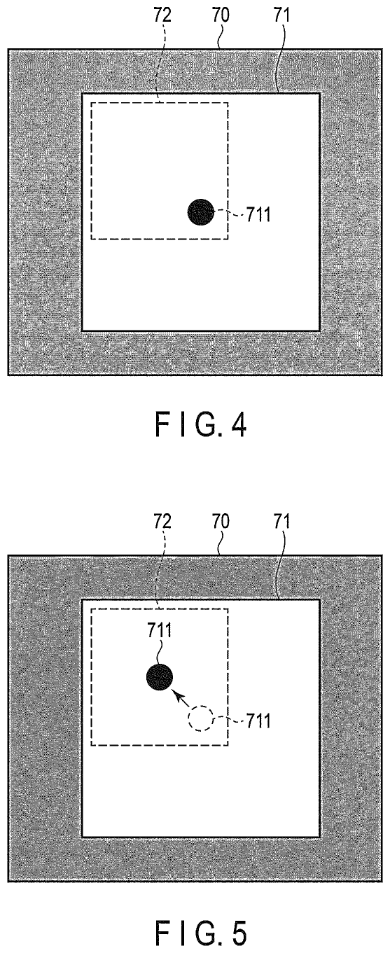

FIG. 4 is a diagram explaining the range displayed by the display 42 in the X-ray imaging. The detector setting range 70 is an area in which the X-ray detector 13 is placed. The irradiation range 71 is an area actually irradiated with X-rays when the irradiation range is narrowed by the X-ray diaphragm. In response to an X-ray emitted from the X-ray tube, the X-ray irradiating the irradiation range 71 is detected by the X-ray detector 13, and an X-ray image of the irradiation range 71 is displayed on the display screen of the display 42. The gray area in FIG. 4 shows that the area is shielded by the X-ray diaphragm.

The user selects one fixed field-of-view size from predefined fixed discrete field-of-view sizes. After the fixed field-of-view size is selected, the processing circuitry 44 determines the irradiation range 71 based on the selected fixed field-of-view size (step S101). At this time, the central position 711 of the irradiation range 71 is set in such a manner that it matches the central position of the detector setting range 70. For this reason, the irradiation range 71 has the same central position as the detector setting range 70, and has a field-of-view size that is defined by the selected fixed field-of-view size.

Next, the processing circuitry 44 causes the display 42 to display the X-ray image generated by limiting the X-ray irradiation range 71 (step S102). At this time, the processing circuitry 44 causes the X-ray tube to emit X-rays through realization of the X-ray control function 443. At this time, the driving of the X-ray diaphragm is controlled in such a manner that only the irradiation range 71 is irradiated with X-rays, and only the irradiation range 71 is irradiated with X-rays.

Next, the processing circuitry 44 generates, through realization of the image generation function 444, an X-ray image based on a signal that is read from the X-ray detector 13.

Next, the processing circuitry 44 causes, through realization of the display control function 445, the display 42 to display the generated X-ray image. At this time, the X-ray image with the enlarged irradiation range 71 is displayed on the display screen of the display 42.

During the X-ray imaging, the X-ray diagnosis apparatus 1 can update the X-ray image to be displayed on the display screen of the display 42 to an X-ray image of the position that the user wants to check, through the operation at the central position controller 432 or the field-of-view size controller 433. Herein, an operation and processing for the case where the user wants to check the X-ray image of the target position 72 shown in FIG. 4 will be described as an example. The target position 72 is an area that the user presently desires to check, for example the position near the device at the time when fluoroscopy imaging is performed. If the user wants to check the X-ray image of the target position 72, the user first moves the central position 711 of the display screen on which the irradiation range 71 is displayed to the center of the target position 72 as shown in FIG. 5 by operating the central position controller 432.

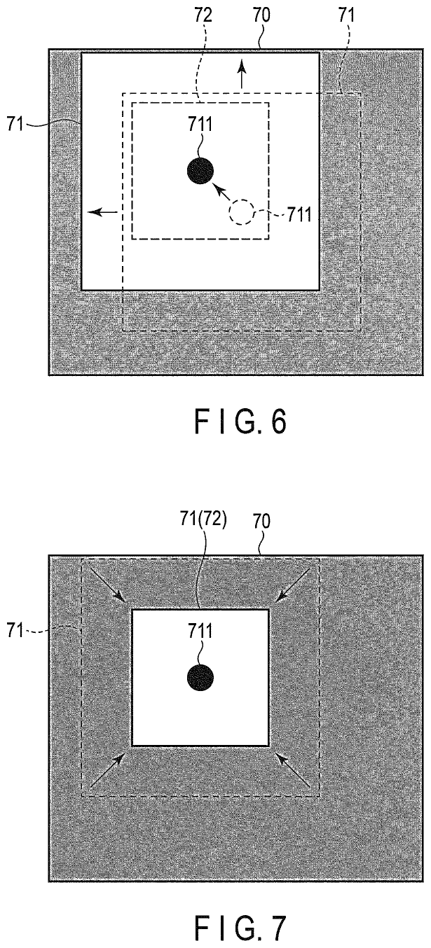

After the operation of changing the central position 711 by the central position controller 432 (Yes in step S103), the processing circuitry 44 changes, through realization of the display control function 445, the irradiation range 71 in accordance with the change of the central position 711. At this time, the irradiation range 71 is changed so that the changed central position 711 is at the center of the field of view, while the selected fixed field-of-view size is being maintained (step S104). As a result, the irradiation range 71 moves in parallel, as shown in FIG. 6.

After the irradiation range 71 is changed, the processing circuitry 44 updates the displayed image (step S105). At this time, through realization of the X-ray control function 443, the image generation function 444, and the display control function 445, the processing circuitry 44 controls the driving of the X-ray diaphragm, irradiates only the updated irradiation range 71 with X-rays to generate an X-ray image, and updates the displayed image to the generated X-ray image.

Next, the user operates the field-of-view size controller 433 to change the field-of-view size of the display screen in accordance with the range that the user wants to check. For example, the user changes the field-of-view size of the display screen to an arbitrarily selected size by moving the slide bar shown in FIG. 2, which is provided as the field-of-view size controller 433, for an arbitrarily selected length.

After the operation of changing the field-of-view size by the field-of-view size controller 433 (Yes in step S106), the processing circuitry 44 changes, through realization of the display control function 445, the irradiation range 71 in accordance with the change in the field-of-view size. At this time, the irradiation range 71 is changed to a size that matches the changed field-of-view size, while the central position 711 of the current displayed image is being maintained (step S107). It is thereby possible to reduce the irradiation range 71 so as to match the field-of-view size of the target position 72 that the user wants to check, as shown in FIG. 7.

During the X-ray imaging, the user can display, every time the position that the user wants to check changes, the X-ray image of an arbitrarily selected position on the display 42 by changing the irradiation range 71 to the arbitrarily selected position through the operation at the central position controller 432 or the field-of-view size controller 433. At this time, if the field-of-view size is changed, the field-of-view can be zoomed in or zoomed out while the central position 711 of the currently displayed irradiation range 71 is being maintained; therefore, it is easy to perform an operation of adapting the field-of-view range of the displayed image to the target position 72. Thus, the user can easily check the desired position.

The processing circuitry 44 performs the processing from step S103 through step S108 every time an operation is performed in the central position controller 432 or the field-of-view size controller 433 until the X-ray imaging is finished, so that the X-ray image being displayed is updated to an X-ray image of the updated target position 72.

In the following description, advantageous effects of the X-ray diagnosis apparatus 1 according to the present embodiment will be described.

The X-ray diagnosis apparatus 1 according to the present embodiment includes an X-ray tube for generating X-rays, the X-ray detector 13 for detecting X-rays, the first operator for controlling the central position of a displayed image being displayed on the display 42, and the second operator for controlling the field-of-view size of the displayed image displayed on the display 42. The first operator is the central position controller 432 of FIG. 2, for example. The second operator is the field-of-view size controller 433 of FIG. 2, for example. The first operator and the second operator are provided in the console apparatus 40. The console apparatus 40 of the X-ray diagnosis apparatus 1 can generate an X-ray image based on X-rays detected by the X-ray detector 13, cause the display 42 to display the generated X-ray image, and change the field-of-view size of the displayed image in response to an operation at the second operator, while the central position of the current displayed image is being maintained. Furthermore, the X-ray diagnosis apparatus 1 further includes an X-ray diaphragm for limiting the X-ray irradiation range, and controls the irradiation range in accordance with an operation at the first operator or the second operator.

For example, suppose the user performs an operation to enlarge (zoom out) the field-of-view size or an operation to reduce (zoom in) the field-of-view size at the field-of-view size controller 433, so that the user can check the target position, such as a region of interest, etc. At this time, the X-ray diagnosis apparatus 1 can enlarge (zoom out) or reduce (zoom in) the displayed image in accordance with an operation at the field-of-view size controller 433, while the central position of the currently displayed image is being maintained.

The X-ray diagnosis apparatus 1 of the present embodiment can change the field-of-view size of a displayed X-ray image to a desired size in a stepless manner through an operation at the field-of-view size controller 433. For this reason, the user can freely set the field-of-view size of a displayed X-ray image, without being limited to the predefined fixed discrete field-of-view sizes.

If the field-of-view size is changed, it is possible to zoom in or zoom out the field-of-view size, while the central position 711 of the currently displayed irradiation range 71 is maintained. For this reason, the field-of-view size can be selected only from multiple predefined fixed field-of-view sizes, and it would be therefore easier to align the display range to the target position 72 compared to the case where the field-of-view size can only be selected from the field-of-view size around the central position of the detector setting range 70. Thus, the user can easily check the desired position.

Thus, it is possible to easily perform an operation to align the field-of-view range of a displayed image to a user's desired target position in the X-ray diagnosis apparatus 1 that is capable of freely controlling an X-ray irradiation range in the X-ray detector 13 through an asymmetrical control of the X-ray diaphragm. It is thereby possible to control the field of view freely and to improve examination efficiency.

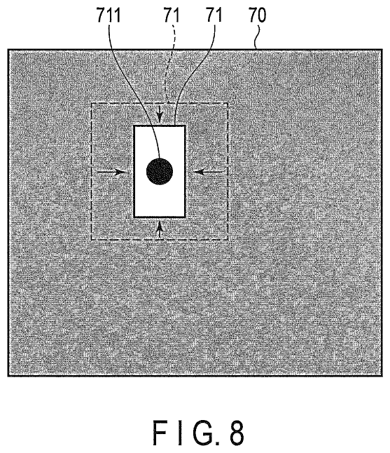

The display range of a displayed image can be further minutely adjusted. In this case, as shown in FIG. 8, for example, a third operator for separately adjusting a degree of opening of the X-ray diaphragm corresponding to the top and the bottom of an X-ray image and a degree of opening of the X-ray diaphragm corresponding to the left and the right of the X-ray image is further provided. The third operator is realized by an operation lever or a button, for example. With an operation of the third operator, it is possible to separately adjust the field-of-view size corresponding to the top and the bottom of the X-ray image of the displayed image and the field-of-view size corresponding to the left and the right of the X-ray image. The processing circuitry 44 controls the X-ray diaphragm based on an operation of the third operator, through realization of the drive control function 442. This allows it to set the irradiation range of the displayed image into a desired shape and to set a further suitable irradiation range. Furthermore, the processing circuitry 44, through realization of the drive control function 442, controls the X-ray diaphragm, which is controlled to move in accordance with the display range of the displayed image, to further move based on an operation of the third operator. It is thereby possible to set a degree of opening of the display range to 100% and to set a degree of opening relative to the display range. FIG. 8 shows a case wherein the field-of-view size in the lateral direction is reduced to 50%, and the field-of-view size in the longitudinal direction is reduced to 70%. The example wherein the degree of opening of the X-ray image in the longitudinal direction and the degree of opening of the X-ray image in the lateral direction are separately adjusted has been described as an example of the third operator; however, the embodiment is not limited to this example, and only the degree of opening in the up and down direction may be separately adjusted. Similarly, only the degree of opening of the X-ray diaphragm in the left and right direction may be separately adjusted.

### Second Embodiment

Next, the second embodiment will be described. The present embodiment is a modification of the configuration of the first embodiment, as will be described below. In the present embodiment, an enlarged image in which a target position is enlarged and an entire image displaying the range including the X-ray irradiation range are displayed on the display 42. This display method is effective when the user wants to enlarge and check the peripheral area of the device while looking at the entire image, for example. In the first embodiment, the X-ray irradiation range is narrowed in accordance with the target position using the X-ray diaphragm; in the present embodiment, on the other hand, the target position is enlarged by image processing, without narrowing the irradiation range. Descriptions of the configurations, operations, and advantageous effects similar to those of the first embodiment will be omitted.

In the present embodiment, the processing circuitry 44 causes, through the display control function 445, one screen of the display 42 to display two images, an entire image of the field-of-view range 73 including the irradiation range 71 and an enlarged image of part of the entire image obtained through image processing. The entire image is an X-ray image including the irradiation range 71, for example. The enlarged image is, for example, an X-ray image of a desired target position, such as a region of interest in the entire image, enlarged by image processing. At this time, the processing circuitry 44 controls the irradiation range in accordance with the field-of-view range of the entire image, and performs image processing to enlarge the target position in relation to the entire image, thereby generating the enlarged image. If the field-of-view size is changed by the second operator, the processing circuitry 44 changes the field-of-view size of the enlarged image to change the enlargement rate, with the central position of the current enlarged image maintained.

The processing circuitry 44 performs, through realization of the display control function 445, two modes, namely, an entire image operating mode of controlling the field-of-view range of an entire image and an enlarged image operating mode of controlling the field-of-view range of an enlarged image. The entire image operating mode corresponds to the first operating mode. The enlarged image operating mode corresponds to the second operating mode. For example, the processing circuitry 44 switches from the entire image operating mode to the enlarged image operating mode in response to an operation at the second operator. The apparatus may include a fourth operator for switching between the entire image operating mode and the enlarged image operating mode. The fourth operator is realized by a switch, a lever, and a button, for example.

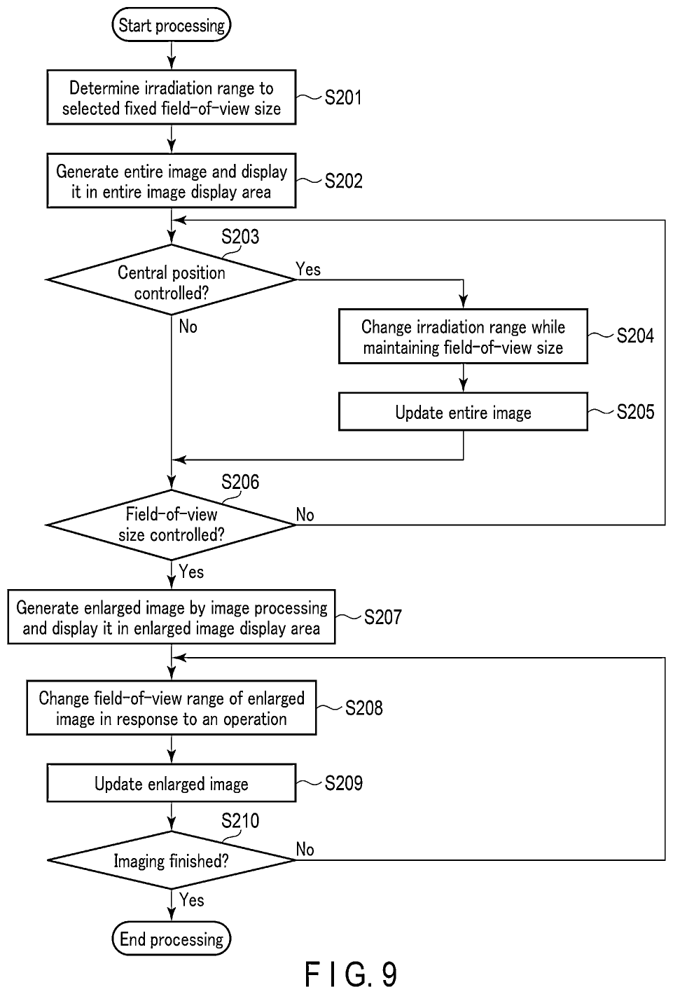

Next, the operation of the X-ray diagnosis apparatus 1 according to the present embodiment is described. FIG. 9 is a flowchart illustrating a processing procedure of the processing performed by the processing circuitry 44 during X-ray imaging. The processing procedure described below is merely an example, and the processing can be changed as far as possible. Omission, replacement, or addition of a step in the processing procedure described hereinafter can be made as appropriate, in accordance with an actual situation where the present embodiment is realized.

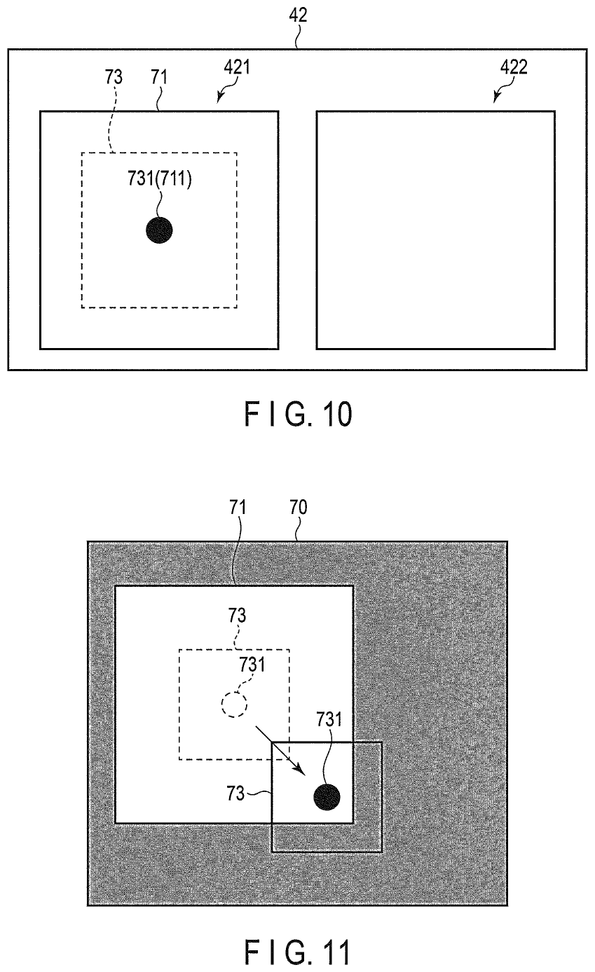

FIG. 10 is a view illustrating an example of a display screen displayed on the display 42 in the present embodiment. In the present embodiment, the entire view display area 421 and the enlarged view display area 422 are displayed in the display 42, as shown in FIG. 10. In the entire view display area 421, an X-ray image having a field-of-view range including the irradiation range 71 is displayed as an entire image. In the enlarged view display area 422, an enlarged X-ray image of part of an image displayed in the entire view display area 421 obtained by image processing is displayed as an enlarged image.

When X-ray imaging is commenced, the X-ray diagnosis apparatus 1 commences X-ray imaging using the imaging apparatus 10 in response to an operation of the start button 431 shown in FIG. 2 by the user. When the X-ray imaging is commenced, the processing circuitry 44 first commences the entire image operating mode of controlling the position or the field-of-view size of the entire image displayed in the entire view display area 421.

In the entire image operating mode, the user first selects one fixed field-of-view size from predefined discrete fixed field-of-view sizes. After the fixed field-of-view size is selected, the processing circuitry 44 determines the irradiation range 71 based on the selected fixed field-of-view size (step S201), and causes the display 42 to display an X-ray image that is generated by limiting the X-ray irradiation range to the irradiation range 71 (step S202), similarly to the processing in step S101 and step S102 of the first embodiment. At this time, the generated X-ray image is displayed in the entire view display area 421 as an entire image.

In the entire image operating mode, the user is able to update the position of the entire image to be displayed in the entire view display area 421 through an operation at the central position controller 432. After an operation to change the central position 711 is performed in the central position controller 432 (Yes in step S203), the processing circuitry 44 changes the irradiation range 71 through realization of the display control function 445 in response to the change of the central position 711. At this time, the irradiation range 71 can be changed so that the changed central position 711 is at the center of the field of view of the entire view display area 421, while the selected fixed field-of-view size is being maintained (step S204). After the irradiation range 71 is changed, the processing circuitry 44 updates the entire image to be displayed in the entire view display area 421 (step S205). Since the processing in step S203 through step S205 is the same as the processing in step S103 through step S105 in the first embodiment, detailed descriptions thereof are omitted.

If the user wants to enlarge part of the entire image displayed in the entire view display area 421, the user causes a transition to the enlarged image operating mode for operating the field-of-view range of the enlarged image to be displayed on the enlarged view display area 422. For example, the entire image operating mode can be switched to the enlarged image operating mode through an operation at the field-of-view size controller 433. A button for switching between the entire image operating mode and the enlarged image operating mode can be separately provided.

During the processing under the entire image operating mode, if an operation at the field-of-view size controller 433 is performed (Yes in step S206), the processing circuitry 44 transitions to the enlarged image operating mode of controlling the position or the field-of-view size of the enlarged image to be displayed in the enlarged view display area 422. Thereafter, the processing circuitry 44 commences, through the display control function 445, the display of the enlarged image on the enlarged view display area 422 (step S207). At this time, as shown in FIG. 10, it is preferable that the position relationship of the field-of-view range of the enlarged image be ascertained by displaying, on the entire image displayed on the entire view display area 421, the field-of-view range 73 of the enlarged image, the central position 731 of the enlarged image, and the central position 711 of the entire image.

In the enlarged image operating mode, the user can change the field-of-view range 73 of the enlarged image in a real-time manner by performing an operation at the central position controller 432 or the field-of-view size controller 433 every change of the target position that the user wants to check. Every operation at the central position controller 432 or the field-of-view size controller 433, the processing circuitry 44 changes the field-of-view range 73 of the enlarged image by the display control function 445 (step S208), and updates the enlarged image to be displayed on the enlarged view display area 422 by performing image processing based on the changed field-of-view range 73.

For example, the user can change the central position 731 of the enlarged image by operating the central position controller 432. At this time, the processing circuitry 44 generates an enlarged image having the changed position as a center and maintaining the field-of-view size of the current enlarged image by performing image processing on the X-ray image displayed on the entire view display area 421, and updates the enlarged image to be displayed on the enlarged view display area 422 to the generated image.

The user can change the field-of-view size of the enlarged image displayed in the enlarged view display area 422 by operating the field-of-view size controller 433. At this time, the processing circuitry 44 generates an enlarged image that maintains the central position 731 of the current enlarged image and has the changed field-of-view size by performing image processing on the entire image, and updates the enlarged image displayed on the enlarged view display area 422 to the generated image. For example, in the state shown in FIG. 6, if the field-of-view size is changed in accordance with the target position 72 through an operation of the field-of-view size controller 433, the enlargement rate of the enlarged image is changed without changing the irradiation range 71 and displayed in the enlarged view display area 422, as shown in FIG. 11.

Furthermore, there may be a case where the field-of-view range 73 of the enlarged image is moved to a position beyond the irradiation range 71 as shown in FIG. 11, by changing the central position or the field-of-view size of the field-of-view range 73 of the enlarged image. In this case, since the part of the field-of-view range 73 of the enlarged image outside the irradiation range 71 is shielded by the X-ray diaphragm, it is necessary to change the irradiation range 71. For this reason, if the field-of-view range 73 of the enlarged image is changed to a position including an area outside the irradiation range 71, the processing circuitry 44 changes, through realization of the drive control function 442, the irradiation range 71 to the range including the field-of-view range 73 of the enlarged image.

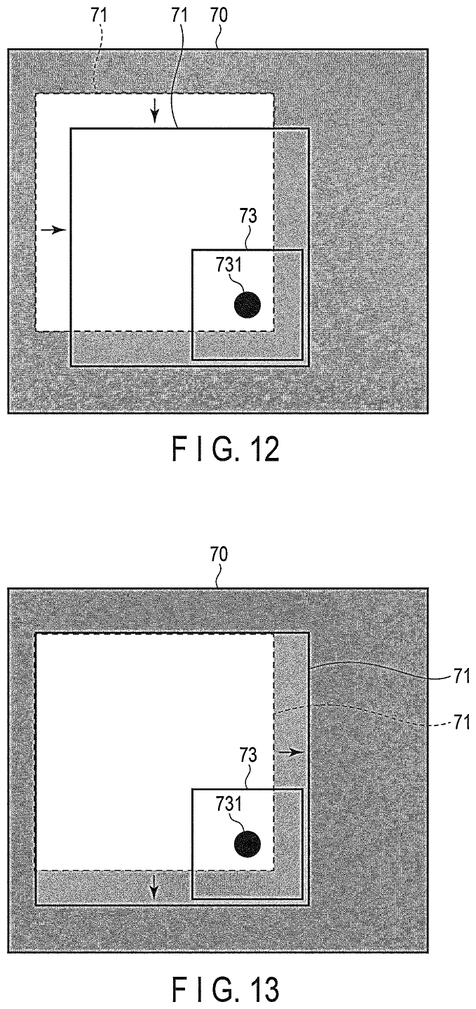

For example, the processing circuitry 44 moves the irradiation range 71 so that the irradiation range 71 includes the field-of-view range 73 of the enlarged image, as shown in FIG. 12. It is thereby possible to irradiate the entire field-of-view range 73 of the enlarged image with X-rays and to generate and display an X-ray image of a part that the user wants to check.

As shown in FIG. 13, the irradiation range 71 may be enlarged so as to include the field-of-view range 73 of the enlarged image. If the irradiation range 71 is changed, the X-ray irradiation range is changed in accordance with the irradiation range 71, and the X-ray diaphragm is driven in accordance with the X-ray irradiation range. It is thereby possible to irradiate the entire field-of-view range 73 of the enlarged image with X-rays and to generate and display an X-ray image of a part that the user wants to check.

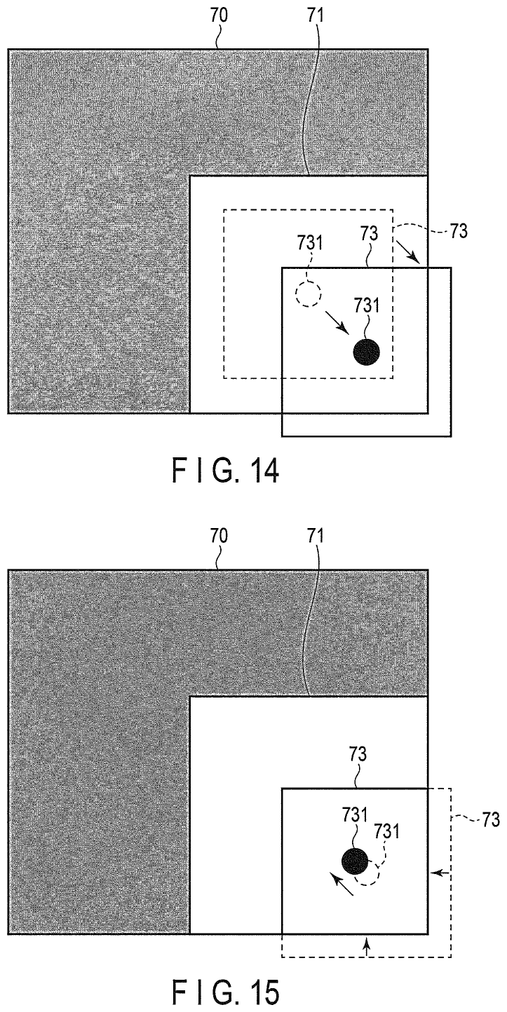

Furthermore, there may be a case where the field-of-view range 73 of the enlarged image is moved to a position beyond the detector setting range 70 as shown in FIG. 14, by changing the central position or the field-of-view size of the field-of-view range 73 of the enlarged image. In this case, the X-ray detector 13 does not exist in the part of the field-of-view range 73 of the enlarged image outside the detector setting range 70. For this reason, the processing circuitry 44 changes, through realization of the display control function 445, the field-of-view range 73 of the enlarged image so as to be included in the setting area of the X-ray detector 13 if the field-of-view range 73 of the enlarged image is changed to the position that includes the area outside the setting area of the X-ray detector 13.

For example, as shown in FIG. 15, the central position 711 or the field-of-view size of the enlarged image is changed so as not to exceed the detector setting range 70. It is thereby possible to irradiate the entire field-of-view range 73 of the enlarged image with X-rays and to generate and display, within the range in which the X-ray detector 13 exists, an X-ray image of a part that the user wants to check.

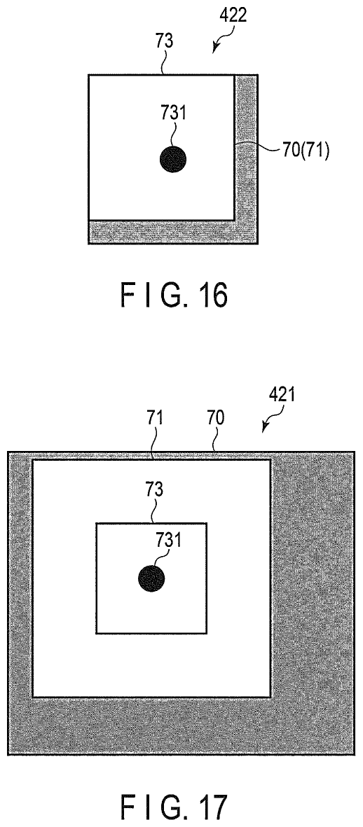

Alternatively, the processing circuitry 44 may show the part of the field-of-view range 73 of the enlarged image outside of the detector setting range 70 in gray as shown in FIG. 16, without changing the field-of-view range 73 of the enlarged image. As shown in FIG. 16, in the enlarged image of the enlarged view display area 422, an X-ray image is displayed in the area overlapping the detector setting range 70, and the area 722 outside of the detector setting range 70 is shown in gray.

If the field-of-view range 73 of the enlarged image exceeds the detector setting range 70, the X-ray detector 13 itself may be moved so that the field-of-view range 73 of the enlarged image is included in the detector setting range 70.

Alternatively, an operation of moving the field-of-view range 73 of the enlarged image to a position beyond the irradiation range 71 or the detector setting range 70 may be disabled. If the field-of-view range 73 of the enlarged image reaches the edge of the irradiation range 71 or the detector setting range 70, the user may be notified by sound or a screen display that the operation has the limit.

The processing circuitry 44 updates the enlarged image to be displayed on the enlarged view display area 422 by repeatedly performing the process from step S208 to step S209 every operation at the central position controller 432 or the field-of-view size controller 433, until the X-ray imaging is finished (No in step S210).

In the following description, advantageous effects of the X-ray diagnosis apparatus 1 according to the present embodiment will be described.

The X-ray diagnosis apparatus 1 of the present embodiment can cause the display 42 to display the enlarged image including the irradiation range 71 and the enlarged image, which is a partial enlarged image of the entire image. For example, the entire image is an X-ray image including the entire irradiation range 71, and the enlarged image is an X-ray image displaying an enlarged target position included in the entire image. The entire image is generated by controlling the irradiation range 71 in accordance with the field-of-view range of the entire image, and the enlarged image is generated through image processing on the entire image.

The X-ray diagnosis apparatus 1 performs the entire image operating mode of controlling the field-of-view range of the entire image and the entire image operating mode of controlling the field-of-view range 73 of the enlarged image. The entire image operating mode corresponds to a first operating mode, and the enlarged image operating mode corresponds to a second operating mode. In the entire image operating mode, the first operating mode functions as an interface of controlling the central position of the entire image, and in the enlarged image operating mode, it functions as an interface of controlling the central position of the enlarged image. The second operator functions as an interface of switching from the entire image operating mode to the enlarged image operating mode, and functions as an interface of controlling the field-of-view size of the enlarged image in a stepless manner in the enlarged image operating mode.

The user can easily know the position of the field-of-view range 73 of the enlarged image in the irradiation range 71 by checking the enlarged image of an enlarged target position and the enlarged image including the entire irradiation range 71 at the same time. For this reason, it is possible to efficiently perform the operation of adjusting the field-of-view range 73 of the enlarged image to a user desired target position. It is thereby possible to control the field of view freely and to improve examination efficiency further.

In the present embodiment, if an operation of changing the field-of-view size is input in the second operator in both the entire image operating mode and the enlarged image operating mode, the displayed image can be enlarged (zoomed out) or reduced (zoomed in) while the current center position is maintained, and the field-of-view size of the displayed image can be changed to a discretionarily selected stepless size. For this reason, the user can freely set the field-of-view size of the displayed image without being restricted to the predefined discrete fixed field-of-view sizes.

An X-ray image including the entire detector setting range 70 may be displayed in the entire view display area 421 as an entire image, as shown in FIG. 17. In the detector setting range 70 shown in FIG. 17, an X-ray image is displayed in the irradiation range 71 and the area shielded from X-rays by the X-ray diaphragm is shown in gray.

In the entire view display area 421, a schematic diagram of a simple diagram illustrating a positional relationship between the detector setting range 70, the irradiation range 71, the field-of-view range 73 of the enlarged image, and the central position 731 of the field-of-view range 73 may be displayed. At this time, an image obtained by a visible radiation camera, a schema simulating a current arrangement with use of a camera, etc., or an actual X-ray image may be displayed in the background of such a schematic diagram. In this case, it is possible to ascertain a positional relationship more accurately. As an X-ray image displayed in the background, an LIH image generated by the LIH (last image hold) function may be used. In this case, if an operation of enlarging (zooming out) or reducing (zooming in) the LIH image displayed in the entire view display area 421 is performed, a field-of-view range slated to be changed as a result of the operation is cropped from the LIH image in advance and is displayed in the enlarged view display area 422. This process makes it easy to know what kind of image can be obtained as a result of the operation and allows the user to adjust the position for displaying the image without X-ray irradiation.

In the enlarged image operating mode, a timing of performing image processing may be adjusted as appropriate. For example, an image generated using a simple digital zoom is displayed in the enlarged view display area 422 until the field-of-view size of the enlarged image is determined, and image processing for enlarging the target position may be performed after the field-of-view size of the enlarged image is determined.

A timing of partial reading performed on the X-ray detector 13 may be adjusted as appropriate. For example, a suitable partial reading may be applied when fluoroscopy is cut off.

Types and conditions of image processing performed on an entire image and an enlarged image may be changed.

### (Modifications)

In the foregoing embodiments, an example of changing the central position of a displayed image using the first operator or the second operator after a fixed field-of-view size is selected; however, the step of selecting the fixed field-of-view size may be omitted. In this case, when an X-ray image of a selected fixed field-of-view size is displayed, a generated X-ray image having the detector setting range 70 as the irradiation range 71 is displayed instead.

To compensate the heel effect, X-ray conditions may be controlled so that the peak of the X-ray intensity distribution overlaps the central position of a displayed image.

The target position 72 and the field-of-view range 73 of an enlarged image can be used as a range used for calculating dose setting in an automatic brightness control (ABC) function or an auto-exposure control (AEC) function. In this case, more appropriate dose setting can be performed by performing dose setting on an area that the user truly wants to see. The target position 72 and the field-of-view range 73 of an enlarged image that are adjusted using a result of segmentation processing may be used as a range used for calculating dose setting.

According to at least one embodiment described above, it is possible to provide a field-of-view control in a free and efficient way during X-ray imaging.

Regarding the foregoing embodiments, the appendage of the following is disclosed as one aspect and selective features of the invention.

### (Additional Note 1)

An X-ray diagnostic apparatus includes:
an X-ray tube that generates X-rays;
an X-ray detector that detects the X-rays;
an image generating unit that generates an X-ray image based on the detected X-rays;
a display controller that causes a display to display the X-ray image;
a first operation unit that receives an operation of inputting a central position for a displayed image to be displayed on the display; and a second operation unit that receives an operation of inputting a field-of-view size of the displayed image in a stepless manner.

If the field-of-view size is changed in the second operation unit, the display controller changes the field-of-view size of the displayed image, while a central position of a current displayed image is being maintained.

### (Additional Note 2)

The X-ray diagnosis apparatus may further include an X-ray diaphragm that limits an irradiation range of the X-rays, and a drive controller that controls driving of the X-ray diaphragm, and the drive controller may control the irradiation range in response to an operation at the first operation unit or at the second operation unit.

### (Additional Note 3)

The X-ray diaphragm includes of four diaphragm blades, and the drive controller may control the irradiation range by moving the positions of the four diaphragm blades separately.

### (Additional Note 4)

The X-ray diagnosis apparatus may further include a third operation unit that receives an operation of inputting a degree of opening of the X-ray diaphragm corresponding to the top and the bottom of the X-ray image and a degree of opening of the X-ray diaphragm corresponding to the left and the right of the X-ray image, and the drive controller may control the X-ray diaphragm in response to an operation at the third operation unit.

### (Additional Note 5)

The second operation unit may be an operation unit that allows a reception of a move operation that is input by a user, and may receive an input of the field-of-view size based on the move operation that is input by the user.

### (Additional Note 6)

The second operation unit may be an operation unit that allows a reception of a move operation that is input by a user, may receive an input regarding a reduction of the field-of-view size in accordance with a move operation that is input by the user for a movement in a first direction, and may receive an input regarding an enlargement of the field-of-view size in accordance with a move operation that is input by the user for a movement in a direction opposite to the first direction.

### (Additional Note 7)

The second operation unit may be a slide bar that receives an input of a field-of-view size in accordance with an amount of movement of the slide bar by a user.

### (Additional Note 8)

The second operation unit may further receive an operation of canceling an input of a field-of-view size in addition to the operation of inputting a field-of-view size.

### (Additional Note 9)

The second operation unit may be a button provided in the first operation unit, and the display controller may determine an amount of change in the field-of-view size of the displayed image in accordance with how the button is pressed by the user and an input operation to the first operation unit.

### (Additional Note 10)

The display controller may cause the display to display an entire image including the X-ray irradiation range and an enlarged image in which a part of the entire image is enlarged.

### (Additional Note 11)

The X-ray diagnosis apparatus may further include an X-ray diaphragm that limits an irradiation range of the X-rays and a drive controller that controls driving of the X-ray diaphragm, and the drive controller may control the irradiation range in accordance with a field-of-view range of the entire image, and the display controller may generate the enlarged image by image processing on the entire image.

### (Additional Note 12)

The display controller may perform a first operating mode of controlling the field-of-view range of the entire image and a second operating mode of controlling the field-of-view range of the enlarged image.

### (Additional Note 13)

The display controller may switch from the first operating mode to the second operating mode in response to a performance of an operation at the second operation unit.

### (Additional Note 14)

If the field-of-view range of the enlarged image may be changed to a position that includes an area outside of the irradiation range, the drive controller may change the irradiation range to a range that includes the field-of-view range of the enlarged image.

### (Additional Note 15)

If the field-of-view range of the enlarged image is changed to a position that includes an area outside of the setting area of the X-ray detector, the display controller may change the field-of-view range of the enlarged image so as to be included in the setting range of the X-ray detector.

### (Additional Note 16)

A console apparatus includes:
a display controller that causes a display to display an X-ray image;
a first operation unit that receives an operation of inputting a central position for a displayed image, which is an X-ray image to be displayed on the display; and
a second operation unit that receives an operation of inputting a field-of-view size of the displayed image in a stepless manner,
wherein the display controller changes the field-of-view size of the displayed image in response to an operation at the second operation unit, while the central position of the current displayed image is being maintained.

### (Additional Note 17)

The console apparatus may further include an X-ray diaphragm that limits an irradiation range of X-rays and a drive controller that controls driving of the X-ray diaphragm, and the drive controller may control the irradiation range in response to an operation at the first operation unit or at the second operation unit.

### (Additional Note 18)

The second operation unit may be an operation unit that allows a reception of a move operation that is input by a user, and may receive an input of the field-of-view size based on the move operation that is input by the user.

### (Additional Note 19)

The second operation unit may be an operation unit that allows a reception of a move operation that is input by a user, and may receive an input regarding a reduction of the field-of-view size in accordance with a first-direction move operation that is input by the user, and may receive an input regarding an enlargement of the field-of-view size in accordance with a move operation that is input by a user for a movement in a first direction.

### (Additional Note 20)

The second operation unit may be a slide bar that receive an input of a field-of-view size in accordance with an amount of movement of the slide bar by a user.

### (Additional Note 21)

The second operation unit may further receive an operation of canceling an input of a field-of-view size in addition to the operation of inputting a field-of-view size.

### (Additional Note 22)

The second operation unit may be a button provided in the first operation unit, and the display controller may determine an amount of change in the field-of-view size of the displayed image in accordance with how the button is pressed by the user and an input operation to the first operation unit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnosis apparatus comprising:
an X-ray tube that generates X-rays;
an X-ray detector that detects the X-rays;
processing circuitry configured to generate an X-ray image based on the detected X-rays, and causes a display to display the X-ray image;
a first operator that receives an operation of inputting a central position for a displayed image to be displayed on the display; and
a second operator that receives an operation of inputting a field-of-view size of the displayed image in a stepless manner, wherein
the processing circuitry is configured to change the field-of-view size of the displayed image in response to an operation at the second operator, while the central position of a current displayed image is being maintained.

2. The X-ray diagnosis apparatus according to claim 1, further comprising an X-ray diaphragm that limits an irradiation range of the X-rays, wherein
the processing circuitry is configured to control driving of the X-ray diaphragm and control the irradiation range in accordance with an operation at the first operator or the second operator.

3. The X-ray diagnosis apparatus according to claim 2, wherein
the X-ray diaphragm includes of four diaphragm blades,
the processing circuitry is configured to control the irradiation range by separately moving positions of the four diaphragm blades.

4. The X-ray diagnosis apparatus according to claim 2, further comprising a third operator that receives an operation of separately inputting a degree of opening of the X-ray diaphragm corresponding to a top and a bottom of the X-ray image and a degree of opening of the X-ray diaphragm corresponding to a right and a left of the X-ray image, wherein
the processing circuitry is configured to control the X-ray diaphragm based on an operation at the third operator.

5. The X-ray diagnosis apparatus according to claim 2, wherein
the second operator receives a move operation that is input by a user, and an input of the field-of-view size based on a move operation that is input by the user.

6. The X-ray diagnosis apparatus according to claim 2, wherein
the second operator receives a move operation that is input by a user, receives an input relating to a reduction of the field-of-view size in accordance with a move operation that is input by the user for a movement in a first direction, and receives an input relating to an enlargement of the field-of-view size in accordance with a move operation that is input by a user for a movement in a direction opposite to the first direction.

7. The X-ray diagnosis apparatus according to claim 1, wherein
the second operator is a slide bar, and the second operator receives an input of a field-of-view size in accordance with an amount of movement of the slide bar by a user.

8. The X-ray diagnosis apparatus according to claim 2, wherein
the second operator receives an operation of canceling an input of a field-of-view size in addition to the operation of inputting a field-of-view size.

9. The X-ray diagnosis apparatus according to claim 1, wherein
the second operator is a button provided in the first operator,
the processing circuitry is configured to determine an amount of change in the field-of-view size of the displayed image in accordance with how the button is pressed by a user and an input operation to the first operator.

10. The X-ray diagnosis apparatus according to claim 1, wherein
the processing circuitry is configured to cause the display to display an entire image including the X-ray irradiation range and an enlarged image in which a part of the entire image is enlarged.

11. The X-ray diagnosis apparatus according to claim 10 further comprising an X-ray diaphragm that limits the irradiation range of the X-rays, wherein
the processing circuitry is configured to:
control driving of the X-ray diaphragm;
control the irradiation range in accordance with a field-of-view range of the entire image; and
generate the enlarged image by image processing on the entire image.

12. The X-ray diagnosis according to claim 10, wherein
the processing circuitry is configured to perform a first operating mode of controlling a field-of-view range of the entire image and a second operating mode of controlling a field-of-view range of the enlarged image.

13. The X-ray diagnosis apparatus according to claim 12, wherein
the processing circuitry is configured to switch from the first operating mode to the second operating mode in response to a performance of an operation at the second operator.

14. The X-ray diagnosis apparatus according to claim 11, wherein
if a field-of-view range of the enlarged image is changed to a position that includes an area outside of the irradiation range, the processing circuitry is configured to change the irradiation range to a range that includes the field-of-view range of the enlarged image.

15. The X-ray diagnosis apparatus according to claim 11, wherein
if a field-of-view range of the enlarged image is changed to a position that includes an area outside of a setting area of the X-ray detector, the processing circuitry is configured to change the field-of-view range of the enlarged image so that the field-of-view range of the enlarged image is included in the setting range of the X-ray detector.

16. A console apparatus comprising:
processing circuitry configured to cause a display to display an X-ray image;
a first operator that receives an operation of inputting a central position for a displayed image, which is an X-ray image to be displayed on the display; and
a second operator that receives an operation of inputting a field-of-view size of the displayed image in a stepless manner, wherein
the processing circuitry is configured to change the field-of-view size of the displayed image in response to an operation at the second operator, while the central position of the current displayed image is being maintained.

17. The console apparatus of claim 16, further comprising an X-ray diaphragm that limits the irradiation range of the X-rays, wherein
the processing circuitry is configured to control driving of the X-ray diaphragm and control the irradiation range in accordance with an operation at the first operator or the second operator.

18. The console apparatus of claim 17, wherein
the second operator receives a move operation that is input by a user, and an input of the field-of-view size based on the move operation that is input by the user.

19. The console apparatus of claim 17, wherein
the second operator receives a reception of a move operation that is input by a user, receives an input regarding a reduction of the field-of-view size in accordance with a move operation that is input by the user for a movement in a first direction, and receives an input regarding an enlargement of the field-of-view size in accordance with a move operation that is input by the user for a movement in a direction opposite to the first direction.

20. The console apparatus of claim 16, wherein
the second operator is a slide bar and the second operator receives an input of a field-of-view size in accordance with an amount of movement of the slide bar by a user.
